(19) United States
(12) Patent Application Publication
(10) Pub. No.: US 2024/0423701 A1
(43) Pub. Date: Dec. 26, 2024

(54) METHODS AND ABLATION SYSTEMS FOR BASIVERTEBRAL NERVE ABLATION USING SHORT, HIGH VOLTAGE PULSES

(71) Applicant: Boston Scientific Neuromodulation Corporation, Valencia, CA (US)

(72) Inventors: Courtney Johnson, Valencia, CA (US); Brendan Early Koop, Ham Lake, MN (US); Michael Sean Coe, Plymouth, MN (US)

(21) Appl. No.: 18/747,115

(22) Filed: Jun. 18, 2024

Related U.S. Application Data
(60) Provisional application No. 63/522,367, filed on Jun. 21, 2023.

(51) Int. Cl. A61B 18/14; A61B 18/00; A61B 18/16

(52) U.S. Cl. CPC A61B 18/148; A61B 18/16; A61B 2018/0044; A61B 2018/00577; A61B 2018/0075; A61B 2018/00761; A61B 2018/00767; A61B 2018/1467

(57) ABSTRACT

A method for basivertebral nerve ablation includes inserting at least one ablation electrode into a vertebral body of a patient; and applying pulses to the ablation electrode to ablate the basivertebral nerve within the vertebral body, wherein each of the pulses has a duration of no more than 500 microseconds and at least some of the pulses have a voltage of at least 1 kV or generate an electric field of at least 3 kV/cm.

### CROSS-REFERENCE TO RELATED APPLICATIONS

This application claims the benefit under 35 U.S.C. § 119 (e) of U.S. Provisional Patent Application Ser. No. 63/522,367, filed Jun. 21, 2023, which is incorporated herein by reference.

### FIELD

The present disclosure is directed to the area of ablation systems and methods of making and using the systems. The present disclosure is also directed to basivertebral nerve (BVN) ablation using short, high voltage pulses.

### BACKGROUND

Ablation generators and electrodes can be used for pain relief or functional modification. Ablation can be used to treat cardiac arrhythmias, benign tumors, cancerous tumors, and to control bleeding during surgery. Ablation is a safe, proven means of interrupting pain signals, such as those coming from irritated facet joints in the spine, genicular nerves in the knee, and femoral and obturator nerves in the hip. There is a need for new ablation treatments to address pain and other disorders.

### BRIEF SUMMARY

One aspect is a method for basivertebral nerve ablation. The method includes inserting at least one ablation electrode into a vertebral body of a patient; and applying pulses to the ablation electrode to ablate the basivertebral nerve within the vertebral body, wherein each of the pulses has a duration of no more than 500 microseconds and at least some of the pulses have a voltage of at least 2 kV.

Another aspect is a system for basivertebral nerve ablation. The system includes a memory having instructions stored thereon and a processor configured to execute those instructions to perform actions including applying pulses to the ablation electrode to ablate the basivertebral nerve within the vertebral body, wherein each of the pulses has a duration of no more than 500 microseconds and at least some of the pulses have a voltage of at least 2 kV.

A further aspect is a non-transitory computer-readable medium having stored thereon instructions for basivertebral nerve ablation, wherein the instructions, when executed by a processor, perform actions including applying pulses to the ablation electrode to ablate the basivertebral nerve within the vertebral body, wherein each of the pulses has a duration of no more than 500 microseconds and at least some of the pulses have a voltage of at least 2 kV.

Yet another aspect is a method for basivertebral nerve ablation. The method includes inserting at least one ablation electrode into a vertebral body of a patient; and applying pulses to the ablation electrode to ablate the basivertebral nerve within the vertebral body, wherein each of the pulses has a duration of no more than 500 microseconds and at least some of the pulses generate an electric field of at least 3 kV/cm.

A further aspect is a system for basivertebral nerve ablation. The system includes a memory having instructions stored thereon and a processor configured to execute those instructions to perform actions including applying pulses to the ablation electrode to ablate the basivertebral nerve within the vertebral body, wherein each of the pulses has a duration of no more than 500 microseconds and at least some of the pulses generate an electric field of at least 3 kV/cm.

Another aspect is a non-transitory computer-readable medium having stored thereon instructions for basivertebral nerve ablation, wherein the instructions, when executed by a processor, perform actions including applying pulses to the ablation electrode to ablate the basivertebral nerve within the vertebral body, wherein each of the pulses has a duration of no more than 500 microseconds and at least some of the pulses generate an electric field of at least 3 kV/cm.

In at least some aspects, each of the pulses has a voltage of at least 2 kV. In at least some aspects, each of the pulses has a voltage of at least 3 kV. In at least some aspects, at least a plurality of the pulses each have a duration of no more than 100 microseconds. In at least some aspects, at least a plurality of the pulses each have a duration of no more than 1 microsecond.

In at least some aspects, the pulses are separated from each other by no more than 20 milliseconds. In at least some aspects, the pulses are arranged in a plurality of pulse bursts, wherein each of the pulse bursts includes a plurality of the pulses, wherein the pulses bursts are separated from each other by at least 3 microseconds. In at least some aspects, the pulses are monophasic. In at least some aspects, the pulses are multiphasic.

In at least some aspects, the at least one ablation electrode includes a monopolar ablation electrode, the method further includes attaching a return electrode to the patient. In at least some aspects, the at least one ablation electrode includes at least two monopolar or monoelectrode ablation electrodes. In at least some aspects, the at least one ablation electrode includes at least one bipolar ablation electrode. In at least some aspects, the inserting includes inserting at least one of the at least one ablation electrode through a pedicle of the patient and into the vertebral body.

Another aspect is a method for basivertebral nerve ablation. The method includes inserting at least one ablation electrode into a vertebral body of a patient; and applying pulses to the ablation electrode to ablate the basivertebral nerve within the vertebral body, wherein each of the pulses has a duration of no more than 500 microseconds and at least some of the pulses generate an electric field of at least 3 kV/cm.

In at least some aspects, each of the pulses generates an electric field of at least 3.5 kV/cm. In at least some aspects, at least a plurality of the pulses each have a duration of no more than 100 microseconds. In at least some aspects, the pulses are separated from each other by no more than 20 millisecond.

In at least some aspects, the at least one ablation electrode includes a monopolar ablation electrode, the method further including attaching a return electrode to the patient. In at least some aspects, the at least one ablation electrode includes at least two monopolar or monoelectrode ablation electrodes. In at least some aspects, the at least one ablation electrode includes at least one bipolar ablation electrode.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of components of one embodiment of an ablation system with a monopolar or monoelectrode ablation electrode;

FIG. 2 is a schematic side view of components of one embodiment of an ablation system with a bipolar ablation electrode;

FIG. 3 is a schematic side view of components of one embodiment of an adapter for coupling a bipolar ablation electrode to an ablation generator;

FIG. 4 is a schematic diagram of one embodiment of a method of basivertebral nerve (BVN) ablation using an ablation system and a single ablation electrode;

FIG. 5 is a schematic diagram of one embodiment of a method of BVN ablation using an ablation system and two ablation electrodes; and

FIG. 6 is a block diagram of one embodiment of an ablation generator.

### DETAILED DESCRIPTION

The present disclosure is directed to the area of ablation systems and methods of making and using the systems. The present disclosure is also directed to basivertebral nerve (BVN) ablation using short, high voltage pulses.

Many conventional ablation generators for pain management support monopolar electrodes with one conductor and a temperature measurement device, such as a thermocouple. The return path is provided by a ground pad that is attached to the patient's skin. Some ablation generators for pain management also support bipolar ablation in which the conductive tips of two or more separate monopolar electrodes are placed near each other. One electrode supplies power while the other electrode acts as a return. Each electrode requires one channel on the ablation generator.

Examples of ablation generators and ablation systems and methods of making and using the ablation generators and ablation systems can be found at, for example, U.S. Pat. Nos. 8,992,522; 9,039,701; 9,173,676; 9,717,552; 9,956,032; 10,111,703; 10,136,937; 10,136,942; 10,136,943; 10,194,971; 10,342,606; 10,363,063; 10,588,687; 10,631,915; 10,639,098; and 10,639,101; and U.S. Patent Application Publications Nos. 2014/0066917; 2014/081260; 2014/0121658; 2021/0121224; 2021/0236191; 2022/0202484; 2022/0202485; and 2022/0226039 and U.S. Provisional Patent Applications Ser. Nos. 63/413,122 and 63/413,133, all of which are incorporated herein by reference in their entireties. In at least some embodiments, these ablation systems can be modified or adapted to perform pulsed ablation as described herein.

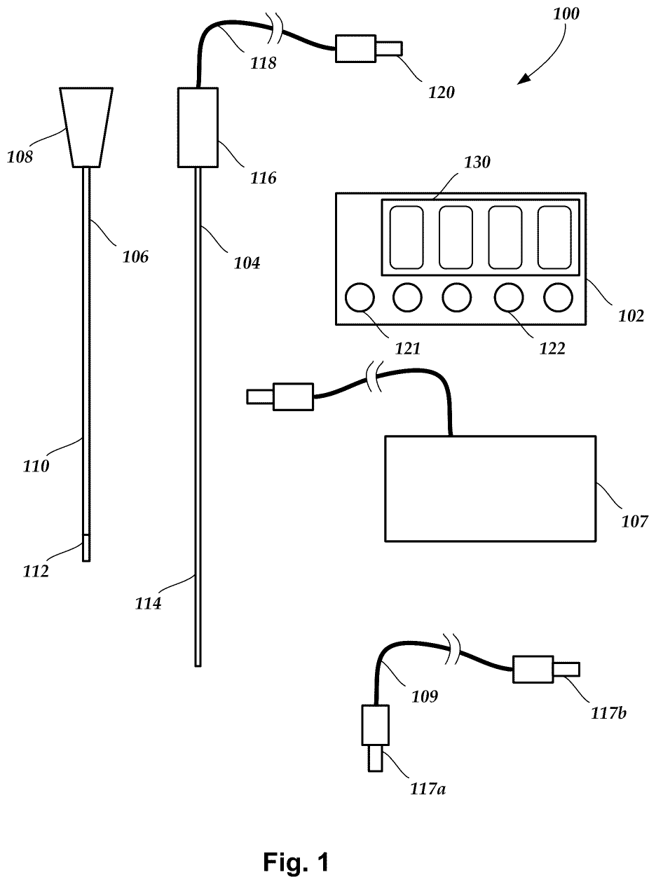

FIG. 1 illustrates one embodiment of an ablation system 100 that includes an ablation generator 102, a monopolar or monoelectrode ablation electrode 104, a cannula 106, a ground pad 107, and an optional extension cable 109. The cannula 106 includes a cannula hub 108, an insulated shaft 110, and an active tip 112. The insulated shaft 110 is hollow with a cannula lumen for receiving the ablation electrode 104. When inserted, the ablation electrode 104 contacts, and energizes, the active tip 112 of the cannula 106 to produce ablation or electrical stimulation. The ablation electrode 104 includes an electrode shaft 114, an electrode hub 116, a cable 118 that is electrically coupled to the electrode shaft 114, and a connector 120 for connecting to a port 122 of the ablation generator 102 to energize the electrode shaft 114 via the cable 118 and connector 120. The optional adapter or extension 109 includes a cable 109 and connectors 117a, 117b for coupling the ablation electrode 104 to the ablation generator 102. It will be recognized that other ablation systems utilize the ablation electrode 104 for ablation instead of, or in addition to, the cannula 106.

The ablation generator 102 can include one or more ports 122 and at least one screen 130. In at least some embodiments, each port 122 is associated with a portion of the screen 130 (or a different screen) and can receive the connector 120 from an ablation electrode 104. Information such as current, voltage, status, or the like or any combination thereof can be displayed on the screen 130. In at least some embodiments, each port 122 corresponds to an independent channel for operating an ablation electrode 104. The ablation generator 102 also includes a ground port 121 for attachment of the ground pad 107.

Basivertebral nerve ablation (BVN) can be used to, for example, treat discogenic back pain or other conditions. The basivertebral nerve is typically ablated using radiofrequency (RF) energy. Typically, the root or central confluence of the major basivertebral nerve intravertebral body branches is located in the near center of the patient's vertebral body (for example, approximately 50%/50% cranial-to-caudal, 50%/50% left-to-right, and 60%-75%/25%-40% anterior-to-posterior.) In at least some embodiments, to access this ablation target a tunnel is created through the vertebral bone and then an ablation electrode is inserted into the vertebral bone for ablation. In at least some embodiments, to form the tunnel the clinician uses a tool to traverse the pedicle of the vertebrae (either left or right side) and then make a turn towards midline. Instead of transpedicular, an extrapedicular approach can be used.

In at least some embodiments, an access tool is used to create a straight tunnel through the pedicle to the vertebral body. Instead of transpedicular, an extrapedicular approach can be used. A tamp tool is then used to create a curved tunnel in the bone. The tamp tools described herein can be used to create a curved tunnel within a hard tough media such as bone. In at least some embodiments, the tamp tool is capable of creating a curved tunnel with enough curve for the various anatomies presented by patient diversity (for example, differences in age, gender, size, or the like or the presence of a disease or disorder, such as scoliosis, which may alter bone shape, density, homogeneity, vertebral form, or the like.) In at least some embodiments, the tamp tool is capable of creating a curved tunnel throughout a range of vertebral levels (for example, at least L3 through S1).

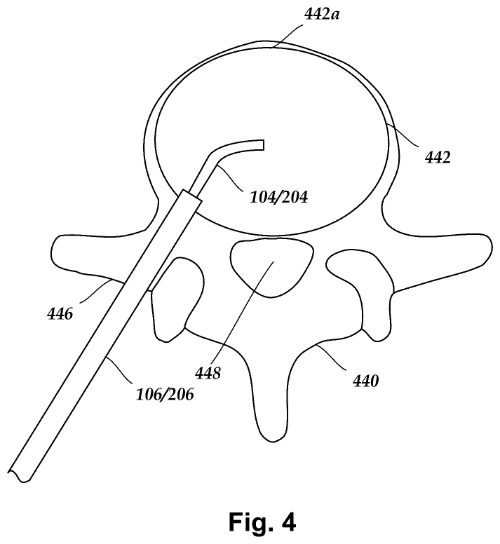

FIG. 4 illustrates, for example, ablation of the basivertebral nerve (BVN) using an ablation system with a monopolar ablation electrode 104 and cannula 106. The BVN resides in the vertebral body 442 of a vertebra 440. Typically, the root or central confluence of the major basivertebral nerve intravertebral body branches is located in the near center of the patient's vertebral body 442 (for example, approximately 50%/50% cranial-to-caudal, 50%/50% left-to-right, and 60%-75%/25%-40% anterior-to-posterior.) In at least some embodiments, to access this ablation target a tunnel is created through the vertebral bone and then the cannula 106 and ablation electrode 104 are inserted into the vertebral bone for ablation. In at least some embodiments, to form the tunnel the clinician uses a tool to traverse the pedicle 335 of the vertebra 440 (either left or right side). In at least some embodiments, the tool, after traversing the pedicle 335, makes a turn towards midline. Instead of transpedicular, an extrapedicular approach can be used. Examples of tools and methods for insertion of an ablation electrode into the vertebral body for BVN treatment can be found in the references cited above.

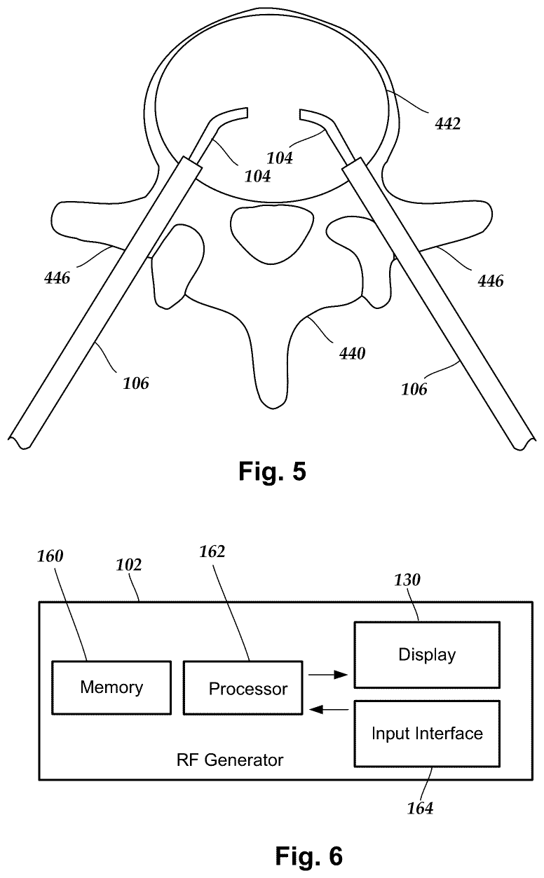

In at least some embodiments, a monopolar ablation electrode 104 and ablation generator 102 can be used to ablate the BVN with a ground pad 107 applied to the patient. FIG. 5 illustrates ablation of the BVN using two monoelectrode ablation electrodes 104 and cannulas 106 (instead of a single monopolar ablation electrode/cannula and ground pad 107). In at least some embodiments, the two cannulas 106 and monoelectrode ablation electrodes 104 are inserted into the vertebral body 442 via the two pedicles 446.

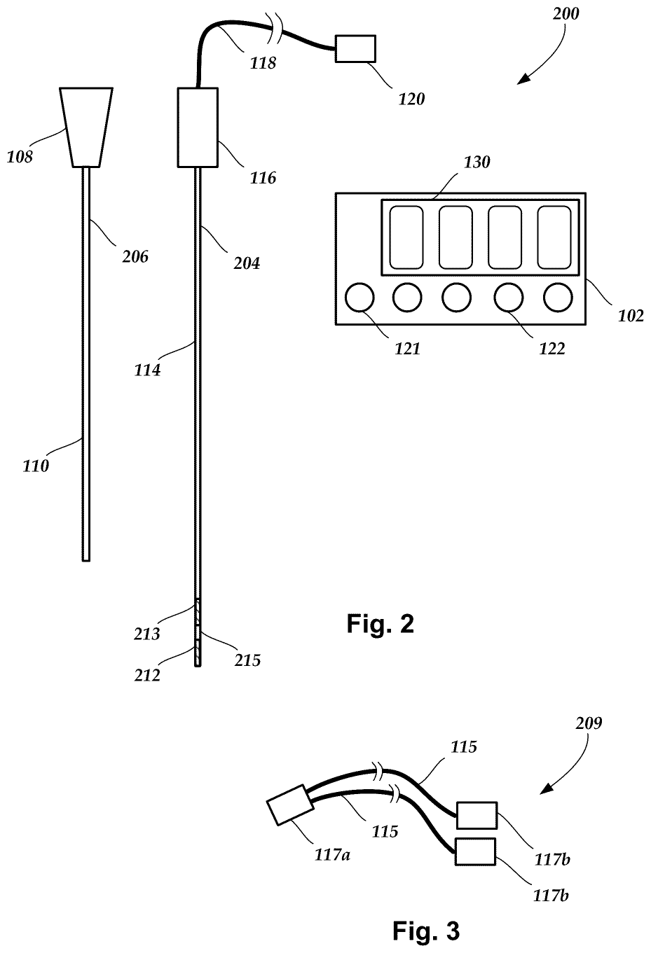

As described herein, an ablation system can include a bipolar electrode (i.e., a component with two ablation electrodes on the same shaft), instead of two or more monoelectrode ablation electrodes. In at least some embodiments, the ablation generator that was previously coupled to monopolar electrodes can be used or adapted for use with a bipolar ablation electrode. FIG. 2 illustrates one embodiment of an ablation system 200 that includes an ablation generator 102, a bipolar ablation electrode 204, and a cannula 206. It should be appreciated that an ablation electrode may be a multipolar ablation electrode having multiple electrodes on the same shaft. It will be recognized that some embodiments of an ablation system can include more or fewer components.

The cannula 206 includes a cannula hub 108 and a cannula shaft 110. The cannula shaft 110 is hollow for receiving the bipolar ablation electrode 204. The bipolar ablation electrode 204 includes an electrode shaft 114, a first electrode 212, a second electrode 213, an insulative material 215 (which may be part of the electrode shaft) separating the first and second electrodes, an electrode hub 116, a cable 118 that is electrically coupled to the electrode shaft 114, and a connector 120 for coupling to at least one port 122 of the ablation generator 102 to energize the first electrode 212 or second electrode 213 (or both) via the cable 118 and connector 120. In at least some embodiments, the length of the insulative material 215 or the separation distance between the first and second electrodes 212, 213 is at least 4, 5, or 6 mm. In at least some embodiments, a separation distance is selected to avoid or reduce barotrauma associated with arcing between the first and second electrodes 212, 213.

The bipolar ablation electrode 204 has two conductors that extend along the cable 118, optionally through the electrode shaft 114, and couple to the first and second electrodes 212, 213, respectively. One conductor is electrically coupled to one of the electrodes (for example, electrode 212) and supplies power to that electrode and the other conductor is electrically coupled to the other one of the electrodes (for example, electrode 213) and acts as a return. In at least some embodiments, the bipolar ablation electrode 204 includes an insulator 215 between the first and second electrodes 212, 213.

At least some ablation generators provide a single channel at each port 122. In at least some embodiments, the bipolar ablation electrode 204 uses a separate channel for each of the two electrodes 212, 213. In at least some embodiments, the ablation system 200 can include an adapter 209, illustrated in FIG. 3, with a connector 117a to connect to the connector 120 of the bipolar ablation electrode 204, two cables 115 that are individually coupled through the connector 117a to a different one of the conductors of the bipolar ablation electrode, and two port connectors 117b for coupling to individual ports 122 of the ablation generator 102. This permits one port 122 to energize one of the electrodes (for example, electrode 212) and another port 122 to act as a return using the other of the electrodes (for example, electrode 213).

FIG. 4 can also illustrate ablation of the basivertebral nerve (BVN) using an ablation system with a bipolar ablation electrode 204 and cannula 206 inserted into the vertebral body 442 via the pedicle 446. In at least some embodiments, the cannula 206 and ablation electrode 204 are inserted into the vertebral body 442 via the pedicle 446.

The ablation systems described above can be used to provide a variety of different treatments. Basivertebral nerve (BVN) ablation can be used to treat disorders or diseases, such as chronic axial low back pain with confirmed Modic type I or II changes. Conventional BVN treatments typically include thermally ablating the BVN with radiofrequency (RF) energy; however, the delivered energy must be controlled to prevent or reduce thermal damage to other tissues, such as the spinal cord. Accordingly, RF-based BVN treatment can require a substantial amount of time to be effective. As an example, according to published clinical information, the average procedure length for BVN ablation is 90 minutes which includes the performance of two 15 minute, temperature-controlled RF ablations to treat a single degenerated disc. It is desirable to reduce the amount of time for a treatment procedure.

In contrast to conventional thermal ablation, short, high voltage pulses can be used for BVN ablation. Although no particular theory is necessary for practicing the invention, it is believed that the short, high voltage DC pulses, which can generate locally high electric fields, cause electroporation of the nerve cells of the BVN. This results in disruption of the cell by generating pores in the cell membrane. The electroporation of the nerve cells of the BVN produces irreversible damage that results in cell death or destruction. It is believed that, if the applied electric field at the membrane is larger than a threshold value, the electroporation is irreversible and the pores remain open. This allows exchange of material across the membrane and leads to necrosis or cell death.

Because the high voltage pulses are applied for a much shorter time (for example, in a window of no more than 10 seconds), the total amount of localized heating can be much less than for the conventional RF-based BVN ablation. Accordingly, damage to other surrounding tissue is avoided or substantially reduced. This is particularly important for BVN ablation due to the proximity of the BVN to the spinal cord.

Nerve cells are particularly resistant to electroporation. In at least some instances, the nerve cells have a higher threshold to electroporation and require a higher electric field (in at least some embodiments, at least 3800 V/cm) to electroporate than other cells. For example, in at least some instances, the nerve cells require at least nine times the electric field to electroporate than myocardial cells or at least twice the electric field to electroporate than red blood cells, vascular smooth muscle cells, or endothelial cells. There may be concern regarding the use of such high voltage pulses near the spinal cord or other sensitive tissues. In at least some embodiments, the size of the irreversible ablative zone of the electric field can be managed (for example, through electrode design or ablation parameters) for irreversible ablation of the BVN but not surrounding structures.

In at least some embodiments, the voltage of the pulses is at least 2, 2.5, 3, 3.5, 3.8, 4, 4.5, or 5 kV. In at least some embodiments, the pulses generate an electric field of at least 3.5, 3.8, 4, 4.5, or 5 kV/cm. In at least some embodiments, a time window for delivery of a treatment set of short, high voltage DC pulses is no more than 100, 150, 200, 250, 300, 500, or 750 milliseconds or 1, 2, or 5 seconds. In at least some embodiments, a single treatment set of short, high voltage DC pulses is sufficient for BVN ablation. In other embodiments, two, three, four, five, or more treatment sets of short, high voltage DC pulses are used for the BVN ablation. In at least some embodiments, the time between treatment sets is at least 1, 2, 5, 10, 20, 30, 45, or more seconds and may be measured in minutes or may be longer. In at least some embodiments, the time between treatment sets may allow for heat dissipation or to charge the capacitors of the ablation generator or any combination thereof.

A treatment set of pulses includes multiple pulses that are delivered during the treatment time window. The pulses can be monophasic, biphasic, or multiphasic. In at least some embodiments, an interphase time delay for a biphasic or multiphasic pulse is no longer than the duration of one phase of the pulse. In at least some embodiments, the time duration for each of the pulses is no more than 0.5, 1, 5, 10, 20, 50, 100, 200, or 500 nanoseconds or 1, 5, 10, 20, 50, 100, 200, 300, or 500 microseconds. In at least some embodiments, the time duration of each of the pulses is at least 0.5, 1, 5, 10, 20, 50, 100, 200, or 500 nanoseconds or 1, 5, 10, 20, 50, 100, 200, or 300 microseconds. The number of pulses in a treatment set of pulses may be at least 10, 20, 50, 100, 200, 500, 1000 or more.

In at least some embodiments, the pulses are rectangular or any other suitable pulse shape. In at least some embodiments of rectangular or other pulses, the rise or fall periods of the pulse may not result in sharp increases/decreases. Such rise or fall periods may be used to, for example, manage overshoot or ringing effects.

In at least some embodiments, the pulses can be arranged in pulse bursts with each pulse burst containing at least 2, 5, 10, 15, 20, 25, 30, 40, 50, 100, or more pulses. The pulses of the pulse burst are separated from each other by a period that, at least in some instances, is no more than 1, 10, 50, 100, 200, 500, or 1000 microseconds. In at least some embodiments, the pulses of a pulse burst (or pulses that are not in a pulse burst arrangement) are separated from each other by at least 0.5, 1, 10, 20, 50, 100, 200, or 500 microseconds. In at least some embodiments, the pulses of a pulse burst (or pulses that are not in a pulse burst arrangement) are separated from each other by no more than 1, 10, 20, 50, 100, 200, 500, or 1000 microseconds. In some embodiments, each of the pulses in a pulse burst are identical in phase, identical in duration, or both. In other embodiments, at least two of the pulses differ from each other in the number of phases, duration, or both.

In at least some embodiments, the time window includes multiple pulse bursts, for example, at least 2, 4, 5, 10, or more pulse bursts. In some embodiments, each of the pulses bursts is identical in the number of pulses, the period between pulses, or both. In other embodiments, at least two of the pulse bursts may differ from each other in the number of pulses, the period between pulses, or both.

In at least some embodiments, the pulse bursts are separated from each other by a burst period that, at least in some instances, is no more than 3, 10, 50, 100, 200, 500, or 1000 microseconds and is at least 3, 10, 50, 100, 200, or 500 microseconds. In at least some embodiments, the burst period is at least 3, 4, 5, or 10 times longer than the period between the individual pulses of a burst.

In at least some embodiments, there may be higher levels of aggregation of pulses or pulse bursts. U.S. Pat. No. 10,709,502, incorporated herein by reference in its entirety, discloses examples of pulse arrangements for tissue ablation.

In at least some embodiments, the values for the delivery parameters described above are selected based on one or more factors such as, for example, efficacy, treatment duration, treatment outcomes, patient pain, damage to tissues other than the BVN, tissue heating, generation of bubbles in body fluids, skeletal muscle stimulation, arcing, tissue aggregation, or the like or any combination thereof.

In at least some embodiments, one or two ablation electrodes 104/204 are inserted into the vertebral body 442. For example, the ablation electrode(s) 104/204 can be inserted through the pedicle(s) 446. In at least some embodiments, the ablation electrode(s) 104/204 is/are positioned at a particular distance away from the spinal cord 448 to avoid hearing or damage to the spinal cord. In at least some embodiments, the ablation electrode(s) 104/204 is/are positioned nearer an anterior portion 448 of the vertebral body than to the spinal cord 448.

After insertion of the ablation electrode(s) 104/204, short, high voltage pulses are then applied to the BVN to ablate the nerve cells, as described above. After treatment, the ablation electrode(s) 104/204 are removed from the vertebra 440.

FIG. 6 illustrates one embodiment of an ablation generator 102 that includes a memory 160, a processor 162, a display 130, and an input interface 164. It will be understood that any of these components or combination of components can be provided as part of another device that is coupled, or coupleable, to the ablation generator 102. The processor 162 can include any suitable processor including one or more hardware processors that may be local to the user or non-local to the user or other components of the computer. The processor 102 is configured to execute instructions provided to the processor.

Any suitable memory 160 can be used. The memory 160 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computer.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The memory 160 can have instructions stored thereon. The instructions, when executed by the processor 162 can perform actions. Such actions can include, for example, applying pulses to the ablation electrode to ablate the basivertebral nerve within the vertebral body. The instructions may include parameter values, such as pulse duration, pulse voltage, the electric field generated by the pulses, the number of pulses, the separation time between pulses, the number of pulse bursts, the number of pulses in a pulse burst, the separation time between pulse bursts, and the like or any combination thereof.

The display 130 can be any suitable display, such as a monitor, screen, display, or the like. The input interface 164 can be, for example, keys, a touchpad, a keyboard, a mouse, a touch screen, a track ball, a joystick, a voice recognition system, another device that is in communication with the ablation generator 102, or any combination thereof, or the like and can be used by the user to interact with a user interface of the ablation generator 102.

The input interface 164 allows a user to program or alter parameter values of the ablation generator 102 for delivery of the short, high voltage pulses, as described herein. In at least some embodiments, the user can program the ablation generator 102 prior to a procedure. In at least some embodiments, the user can input or alter parameter values during a procedure.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected is:

1. A method for basivertebral nerve ablation, the method comprising
inserting at least one ablation electrode into a vertebral body of a patient; and
applying pulses to the ablation electrode to ablate the basivertebral nerve within the vertebral body, wherein each of the pulses has a duration of no more than 500 microseconds and at least some of the pulses have a voltage of at least 2 kV.

2. The method of claim 1, wherein each of the pulses has a voltage of at least 2 kV.

3. The method of claim 1, wherein each of the pulses has a voltage of at least 3 kV.

4. The method of claim 1, wherein at least a plurality of the pulses each have a duration of no more than 100 microseconds.

5. The method of claim 1, wherein at least a plurality of the pulses each have a duration of no more than 1 microsecond.

6. The method of claim 1, wherein the pulses are separated from each other by no more than 20 milliseconds.

7. The method of claim 1, wherein the pulses are arranged in a plurality of pulse bursts, wherein each of the pulse bursts comprises a plurality of the pulses, wherein the pulses bursts are separated from each other by at least 3 microseconds.

8. The method of claim 1, wherein the pulses are monophasic.

9. The method of claim 1, wherein the pulses are multiphasic.

10. The method of claim 1, wherein the at least one ablation electrode comprises a monopolar ablation electrode, the method further comprising attaching a return electrode to the patient.

11. The method of claim 1, wherein the at least one ablation electrode comprises at least two monopolar or monoelectrode ablation electrodes.

12. The method of claim 1, wherein the at least one ablation electrode comprises at least one bipolar ablation electrode.

13. The method of claim 1, wherein the inserting comprises inserting at least one of the at least one ablation electrode through a pedicle of the patient and into the vertebral body.

14. A method for basivertebral nerve ablation, the method comprising
inserting at least one ablation electrode into a vertebral body of a patient; and
applying pulses to the ablation electrode to ablate the basivertebral nerve within the vertebral body, wherein each of the pulses has a duration of no more than 500 microseconds and at least some of the pulses generate an electric field of at least 3 kV/cm.

15. The method of claim 14, wherein each of the pulses generates an electric field of at least 3.5 kV/cm.

16. The method of claim 14, wherein at least a plurality of the pulses each have a duration of no more than 100 microseconds.

17. The method of claim 14, wherein the pulses are separated from each other by no more than 20 milliseconds.

18. The method of claim 14, wherein the at least one ablation electrode comprises a monopolar ablation electrode, the method further comprising attaching a return electrode to the patient.

19. The method of claim 14, wherein the at least one ablation electrode comprises at least two monopolar or monoelectrode ablation electrodes.

20. The method of claim 14, wherein the at least one ablation electrode comprises at least one bipolar ablation electrode.
